(19) United States
(12) Patent Application Publication
(10) Pub. No.: US 2004/0158314 A1
(43) Pub. Date: Aug. 12, 2004

(54) Ribbon-type vascular prosthesis having stress-relieving articulation and methods of use

(75) Inventor: Michael Hogendijk, Palo Alto, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: NovoStent Corporation, Santa Clara, CA (US)

(21) Appl. No.: 10/772,840

(22) Filed: Feb. 4, 2004

Related U.S. Application Data
(63) Continuation-in-part of application No. 10/342,427, filed on Jan. 13, 2003.
(60) Provisional application No. 60/436,516, filed on Dec. 24, 2002.

(51) Int. Cl. A61F 2/06

(52) U.S. Cl. 623/1.15

(57) ABSTRACT

A vascular prosthesis is provided having a self-expanding radial distal section joined to a ribbon-type helical section by a stress-relieving articulation. The stress-relieving articulation preferably comprises first and second connection members, each having a hinge and interconnected between the distal and helical sections, proximal ends of the first and second connection members also interconnected by a hinge, so that the articulation permits relatively independent rotation and compression of the distal and helical sections.

### REFERENCE TO RELATED APPLICATIONS

This application is a continuation-in-part of U.S. patent application Ser. No. 10/342,427, filed Jan. 13, 2003, which claims priority from U.S. provisional patent application Serial No. 60/436,516, filed Dec. 24, 2002.

### FIELD OF THE INVENTION

The present invention relates to an implantable vascular ribbon-type prosthesis having a helical section and at least one anchor section, wherein the anchor section is joined to the helical section by a stress-relieving redundant articulation.

### BACKGROUND OF THE INVENTION

Today there are a wide range of intravascular prostheses on the market for use in the treatment of aneurysms, stenoses, and other vascular irregularities. Balloon expandable and self-expanding stents are well known for restoring patency in a stenosed vessel, e.g., after an angioplasty procedure, and the use of coils and stents are known techniques for treating aneurysms.

Previously-known self-expanding stents generally are retained in a contracted delivery configuration using an outer sheath, then self-expand when the sheath is retracted. Such stents commonly have several drawbacks, for example, the stents may experience large length changes during expansion (referred to as “foreshortening”) and may shift within the vessel prior to engaging the vessel wall, resulting in improper placement. Additionally, many self-expanding stents have relatively large delivery profiles because the configuration of their struts limits further compression of the stent. Accordingly, such stents may not be suitable for use in smaller vessels, such as cerebral vessels and coronary arteries.

Other drawbacks associated with the use of coils or stents in the treatment of aneurysms is that the devices, when deployed, may have a tendency to straighten or otherwise remodel a delicate cerebral vessel, which may cause further adverse consequences. Moreover, such devices may not adequately reduce blood flow from the cerebral vessel into the sac of the aneurysm, which may increase the likelihood of rupture. Generally, if a greater surface area is employed to cover the sac, the delivery profile of the device may be compromised due to the increased surface area, and the device also may be more rigid and cause remodeling of the vessel.

For example, PCT Publication WO 00/62711 to Rivelli describes a stent comprising a helical mesh coil having a plurality of turns and including a lattice having a multiplicity of pores. The lattice is tapered along its length. In operation, the plurality of turns are wound into a reduced diameter helical shape, then constrained within a delivery sheath. The delivery sheath is retracted to expose the distal portion of the stent and anchor the distal end of the stent. As the delivery sheath is further retracted, subsequent individual turns of the stent unwind to conform to the diameter of the vessel wall.

The stent described in the foregoing publication has several drawbacks. For example, due to friction between the turns and the sheath, the individual turns of the stent may bunch up, or overlap with one another, when the delivery sheath is retracted. In addition, once the sheath of the delivery catheter is fully retracted, the turns of a ribbon-type stent may shift within the vessel prior to engaging the vessel wall, resulting in improper placement of the stent. Still further, because the distal portion of the stent may provide insufficient engagement with the vessel wall during subsequent retraction of the remainder of the sheath, ambiguity concerning accuracy of the stent placement may arise.

In view of these drawbacks of previously known devices, it has been proposed in copending and commonly assigned U.S. patent application Ser. No. 10/342,427, filed Jan. 13, 2003, to provide an implantable vascular prosthesis comprising a ribbon-type stent body joined at its distal end to a radially expandable anchor. As described in that application, the radially expandable anchor is deployed first to anchor the distal-most portion of the ribbon-type stent body, thereby enhancing accuracy of placement of the prosthesis.

Although the prosthesis described in the above-mentioned application overcomes many of the drawbacks of previously know ribbon-type stents, it has come to be appreciated that the connection between the helical section and the anchor may experience high levels of stress during deployment of the stent. Such stress levels may exceed the elastic range of the stent material, resulting in a less than optimum deployed configuration, or may even lead to fracture of the stent. Accordingly, it would be desirable to provide a vascular prosthesis having a distal anchor and helical section that are joined by a stress-relieving articulation.

It further would be desirable to provide a ribbon-type stent with distal anchor wherein a stress-relief feature permits a planar hinge element to withstand axial compression in a direction normal to the plane of the hinge element.

It also would be desirable to provide a ribbon-type stent with distal anchor having a stress-relief feature that reduces the risk of creating inelastic strain while permitting transfer of high torsional loads.

It further would be desirable to provide a ribbon-type stent with distal anchor having an articulation that permits distribution of torsional loads over an enlarged area, and further provides some redundancy to ensure that the helical and anchor sections of the stent cannot become uncoupled.

### SUMMARY OF THE INVENTION

In view of the foregoing, it is an object of the present invention to provide a vascular prosthesis having a distal anchor and helical section that are joined by a stress-relieving articulation.

It is another object of this invention to provide a ribbon-type stent with distal anchor wherein a stress-relief feature permits a planar hinge element to withstand axial compression in a direction normal to the plane of the hinge element.

It also is an object of this invention to provide a ribbon-type stent with distal anchor having a stress-relief feature that reduces the risk of creating inelastic strain while permitting transfer of high torsional loads.

It is a further object of the present invention to provide a ribbon-type stent with distal anchor having an articulation that permits distribution of torsional loads over an enlarged area, and which includes some redundant connections that ensure that the helical and anchor sections of the stent cannot become uncoupled.

These and other objects of the present invention are accomplished by providing a vascular prosthesis comprising a ribbon-type helical section joined at its distal end to a radially expandable anchor, wherein the helical section is joined to the anchor by a stress-relieving articulation.

In a preferred embodiment, the vascular prosthesis comprises a self-expanding helical ribbon section joined to a self-expanding anchor portion comprising either a generally zig-zag or cell-like strut configuration, wherein the anchor portion is deployed first to fix the distal-most extremity of the stent within a vessel. In accordance with the principles of the present invention, the helical and distal sections are coupled by at least one connection member having a hinge. More preferably, the helical and distal sections are coupled by at least two connection members, each having a hinge, thereby permitting the distribution of torsional loads over a larger region of the adjoining sections. In addition, the presence of multiple connection members enhances safety of the vascular prosthesis by ensuring that the helical and distal sections cannot become uncoupled.

In one preferred embodiment, the distal anchor comprises a cell-like configuration having substantially straight axially-oriented struts coupled by zig-zag portions. Each zig-zag includes a bend preferably having a “C”-shaped semi-circular configuration. In accordance with this invention, a first connection member includes a substantially straight portion extending from the apex of a bend so that it defines a distal edge of the helical section of the vascular prosthesis, and is aligned with, or disposed at an oblique angle to, a longitudinal axis of the prosthesis. The first connection member further comprises a planar hinge having a “C”-shaped semi-circular portion similar to that of a bend that joins adjacent zig-zag portions of the anchor section.

A second connection member also may be provided that extends from the apex of an adjacent bend of the anchor section, and is affixed at its proximal end to the proximal end of, or some intermediate point of, the distal edge of helical section. The second connection member preferably includes a planar hinge that is substantially similar to that provided in the first connection member. The second connection member distributes torsional loads imposed on the anchor section during deployment of the helical section, and assists in stabilizing the distal edge of the helical section during deployment.

Methods of using the vascular prothesis of the present invention, for example, in the treatment of aneurysm or stenosis, also are provided.

### BRIEF DESCRIPTION OF THE DRAWINGS

Further features of the invention, its nature and various advantages will be more apparent from the accompanying drawings and the following detailed description of the preferred embodiments, in which:

FIGS. 1A-1B are, respectively, side and perspective views of a vascular prosthesis suitable for use with the stress-relieving articulation of the present invention;

FIGS. 2A-2B are, respectively, side and perspective views of an alternative embodiment of vascular prosthesis suitable for use with the stress-relieving articulation of the present invention;

FIG. 3 is a perspective view of a vascular prosthesis including the stress-relieving articulation of the present invention;

FIGS. 4A and 4B are partial side views of connection member of the vascular prosthesis of FIG. 3;

FIG. 5 is a side view of an inner member of a delivery catheter suitable for use with the vascular prosthesis of the present invention;

FIG. 6 is a side view, partly in section, illustrating a vascular prosthesis of the present invention disposed within a delivery catheter including the inner member of FIG. 5; and

FIGS. 7A-7G are side-sectional views showing a method of performing angioplasty and delivering the vascular prosthesis using the delivery catheter of FIG. 6.

### DETAILED DESCRIPTION OF THE INVENTION

The present invention is directed to an implantable vascular prosthesis configured for use in a wide range of applications, such as treating aneurysms, maintaining patency of a vessel following angioplasty or providing controlled delivery of therapeutic agents to a vessel wall. The vascular prosthesis of the present invention comprises a helical ribbon portion joined, at its distal end, to a radially self-expanding anchor portion via a stress-relieving articulation. The articulation provides improved axial flexibility, improved distribution and stabilization of torsional stresses, enhanced safety and accuracy in delivering the stent by reducing the risk of inadvertent axial movement of the helical portion during deployment.

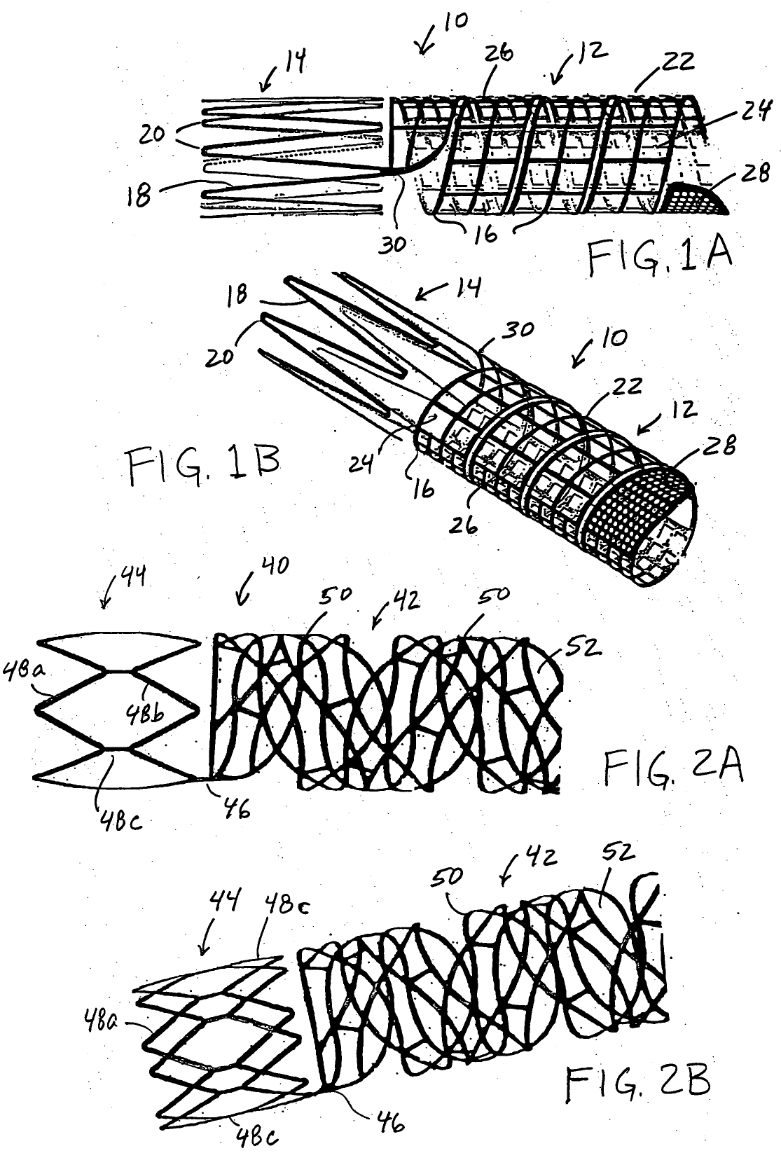

Referring to FIGS. 1A and 1B, a first embodiment of a vascular prosthesis suitable for use with stress-relieving articulation of the present invention is described. Vascular prosthesis 10 is described in copending commonly assigned U.S. patent application Ser. No. 10/342,427, filed Jan. 13, 2003, and comprises helical section 12 and distal section 14, each capable of assuming contracted and deployed states. In FIGS. 1A and 1B, helical section 12 and distal section 14 are each depicted in their respective deployed states.

Vascular prosthesis 10 preferably is formed from a solid tubular member comprising a shape memory material, such as nickel-titanium alloy (commonly known in the art as Nitinol). The solid tubular member then is laser cut, using techniques that are per se known in the art, to a desired deployed configuration, as depicted in FIG. 1. An appropriate heat treatment, per se known in the art, then may be applied to solid regions 16 of vascular prosthesis 10 while the device is held in the desired deployed configuration (e.g., on a mandrel). The treatment of the shape memory material allows vascular prosthesis 10 to self-deploy to the desired deployed configuration, depicted in FIG. 1, for purposes described hereinafter.

Distal section 14 preferably has a generally zig-zag configuration in the deployed state, wherein the zig-zag configuration preferably is formed by laser cutting a solid tube to form a pattern comprising plurality of struts 18 disposed between plurality of bends 20. Distal section 14 is designed to be deployed from a stent delivery catheter first to fix the distal end of the stent at a desired known location within a vessel, whereby subsequent deployment of helical section 12 of the stent may be accomplished with greater accuracy.

Helical section 12 preferably comprises a helical mesh configuration that includes a plurality of substantially flat turns 22. Plurality of turns 22 may include a multiplicity of openings provided in different shapes and sizes, as illustrated by larger rectangular openings 24, smaller rectangular openings 26 and small circular openings 28. The multiplicity of openings are disposed between solid regions 16 of the shape memory material used to form vascular prosthesis 10, although, the configuration of helical section 12 depicted herein is merely for illustrative purposes. Helical section 12 is coupled to distal section 14 at junction 30.

Referring to FIGS. 2A and 2B, an alternative embodiment of a vascular prosthesis suitable for use with stress-relieving articulation of the present invention is described. Vascular prosthesis 40 includes helical section 42 and distal section 44 joined at junction 46. Distal section 44 comprises a radially self-expanding cell-like configuration comprising pair of zig-zags 48a, 48b joined by struts 48c. The cell configuration of FIG. 2 is expected to be more rigid than the single zig-zag configuration of the embodiment of FIG. 1, and hence capable of applying, and withstanding, greater radial force. Helical section 42 preferably comprises a helical ribbon including plurality of turns 50 having multiplicity of openings 52 provided in varying shapes and sizes.

Referring now to FIGS. 3 and 4, vascular prosthesis 60 of the present invention is described. Prosthesis 60 includes distal section 62, similar in design to that of FIG. 2, and helical section 64 joined to the distal section by stress-relieving articulation 66 of the present invention (shown in greater detail in FIGS. 4A and 4B). Distal section 62 comprises a plurality of cells defined by pair of zig-zags 68a, 68b joined by struts 68c, wherein adjacent portions of each zig-zag are coupled by bends 68d that preferably have a “C”-shaped semicircular configuration. Helical section 64 preferably comprises a helical ribbon formed of a multiplicity of spirals 69.

In accordance with the principles of the present invention, stress-relieving articulation 66 comprises first and second connection members 70a and 70b, respectively. Connection member 70a preferably comprises a substantially straight portion that defines a distal edge of helical section 64. This straight portion may be either aligned parallel to, or at an oblique angle α relative to, the longitudinal axis of the prosthesis. As better shown in FIG. 4, connection member 70a includes hinge 72 that is coupled to proximal apex 73 of zig-zag 68b. Hinge 72 preferably has a planar “C”-shaped semicircular configuration similar to that of bends 68d.

Connection member 70b also includes substantially straight portion 73 and hinge 74. Hinge 74 is similar in design to hinge 72, but with opposite concavity relative to hinge 72. Connection member 70b is coupled at one end by hinge 74 to bend 68d of proximal apex 75 of zig-zag 68b, adjacent apex 73, and at the other end to the proximal end of (or at some intermediate location of) connection member 70a by hinge 76. Hinge 76 also preferably has the “C”-shaped configuration of hinges 72 and 74.

Coupling of connection members 70a and 70b via respective hinges 72 and 74 to bends 68d of adjacent apices 73 and 75 of zig-zag 68b allows for relatively independent rotation and compression of the associated zig-zag sections 68c and the connection members relative to the longitudinal axis of the prosthesis (as shown for connection member 70a in dotted line in FIG. 4A). This arrangement, plus coupling the proximal ends of connection members 70a and 70b by hinge 76, is expected to impart minimal stresses on the joint between distal anchor section 62 and helical section 64, while allowing for relatively independent movement of the components of the distal section and the helical section. Potential rotational movements caused by compression of the articulation and its adjacent components are illustrated by arrows in FIGS. 4A and 4B.

The foregoing arrangement advantageously is expected to more uniformly distribute the loads and stresses experienced during deployment of the respective sections of the prosthesis. The use of first and second connection members 70a and 70b also provides a redundant connection between the distal anchor and helical sections that may reduce the risk of inelastic strain or fracture at the junction between the distal anchor and helical section. Furthermore, the presence of connection member 70b coupled to the proximal end (or at some intermediate location) to connection member 70a is expected to stabilize movement of the distal edges of the helical section of the prosthesis during deployment.

It will be understood by one of ordinary skill that the advantages of the foregoing arrangement may be achieved using hinges other than the planar “C”-shaped semi-circular configuration described above. For example, some or all of bend 68d and planar hinges 72, 74 and 76 may be replaced by spiral portions, similar to spirals 69 that define helical section 64 of the prosthesis. As a further alternative, some or all of hinges 72, 74 and 76 may comprise separately formed coil springs that are joined to the distal and helical sections, for example, by welding. Still further, hinges 72 and 74 need not be joined to the apices of the cells of distal section 62, but may instead be joined to the other portions of proximal zig-zag 68b.

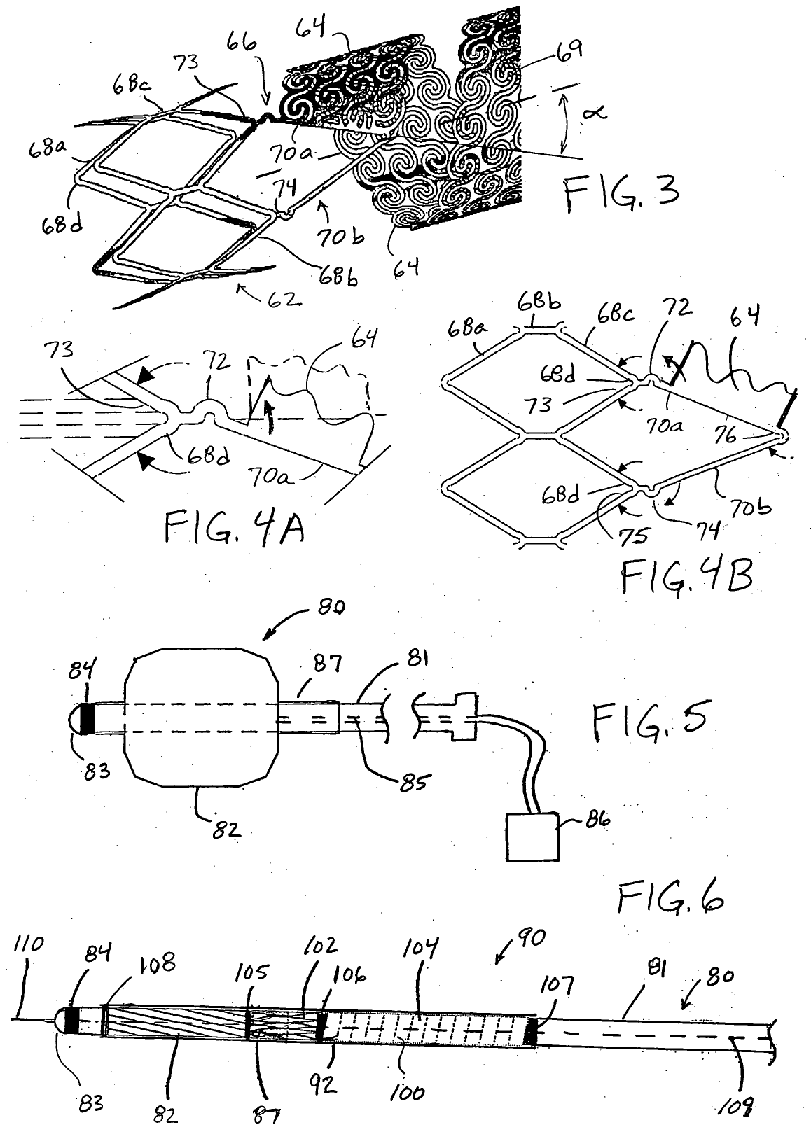

Referring now to FIGS. 5 and 6, a preferred delivery catheter suitable for deploying the vascular prosthesis of the present invention is described. In FIG. 5, inner member 80 of the delivery catheter is depicted, while FIG. 6 shows the inner member carrying a vascular prosthesis of the present invention constrained on inner member 80 by retractable sheath 92.

Referring still to FIG. 5, inner member 80 comprises shaft 81 comprising a sturdy flexible material such as are typically used in catheter manufacture, e.g., polyethylene, and includes balloon 82 disposed adjacent to atraumatic tip 83. Radio-opaque marker 84 is affixed adjacent to tip 83 of shaft 81 to make the distal end of the shaft visible under fluoroscopic imaging. Balloon 82 may be formed from compliant or semi-compliant materials, such as nylon or PEBAX, and is inflated through lumen 85. Lumen 85 may be pressurized with fluid from syringe or inflator 86, which may be selectively coupled to the proximal end of shaft 81, as is known in the art.

Inner member 81 includes polymer layer 87 that engages the distal end of the distal section of vascular prosthesis to prevent it from moving proximally when sheath 92 is retracted. Polymer layer 87 preferably is treated, e.g., by formulation, mechanical abrasion, chemically or by heat treatment, to make the polymer tacky or otherwise enhance the grip of the material. Polymer layer 87 may comprise a proximal shoulder of balloon 82, or alternatively may be formed and applied separately from balloon 82. As a yet further alternative, balloon 82 may be omitted, and polymer layer 87 may be disposed adjacent the distal end of the inner member.

With respect to FIG. 6, delivery catheter 90 is shown pre-loaded with vascular prosthesis 100 of the type shown in FIG. 3, wherein the prosthesis is constrained between inner member 80 and sheath 92. Prosthesis 100 includes distal section 102 that is engaged with polymer layer 87, and helical section 104 that is wrapped to a small diameter around shaft 81 of inner member 80. Sheath 92 restrains vascular prosthesis 100 against shaft 81 of inner member 80 until the sheath is retracted proximally. Balloon 82 is shown deflated and wrapped around shaft 81 of the inner member, in accordance with known techniques.

Sheath 92 is depicted in its insertion configuration, wherein the sheath extends over balloon 82 to a position just proximal of distal end 83. Delivery catheter 90 optionally may include radio-opaque marker bands 105, 106 and 107 disposed, respectively, on inner member 80 beneath the distal and proximal ends of distal section 102 and at the proximal end of helical section 104. Sheath 92 may also include radio-opaque marker 108 disposed adjacent to its distal end. Delivery catheter 90 preferably includes guide wire lumen 109 that enables the delivery catheter to be slidably translated along guide wire 110.

In operation, delivery catheter 90 is advanced along a guide wire into a vessel containing a treatment area. Positioning of the vascular prosthesis relative to the treatment area is confirmed using radio-opaque markers 84 and 105-107. Once the delivery catheter is placed in the desired location, sheath 92 is retracted proximally to permit vascular prosthesis 100 to deploy. Polymer layer 87 grips distal section 102 of stent 100, and prevents distal section 102 from being dragged proximally into engagement with helical section 104 during retraction of sheath 92. Instead, polymer section 87 grips distal section 102 against axial movement, and permits the distal section to expand radially outward into engagement with the vessel wall once the outer sheath is retracted.

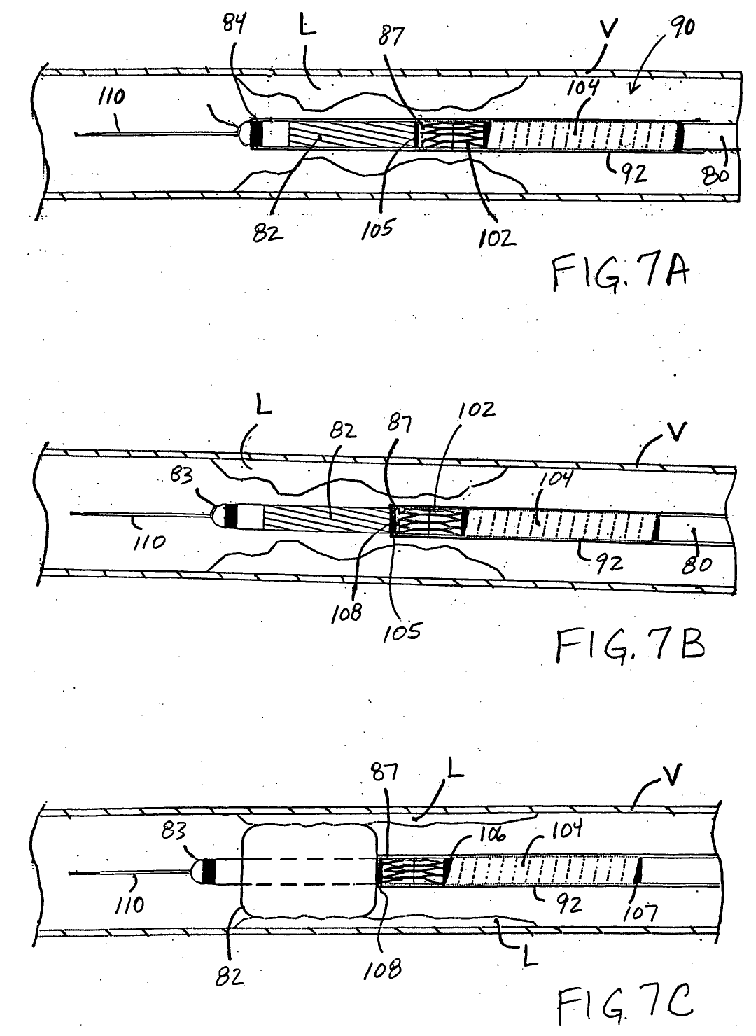

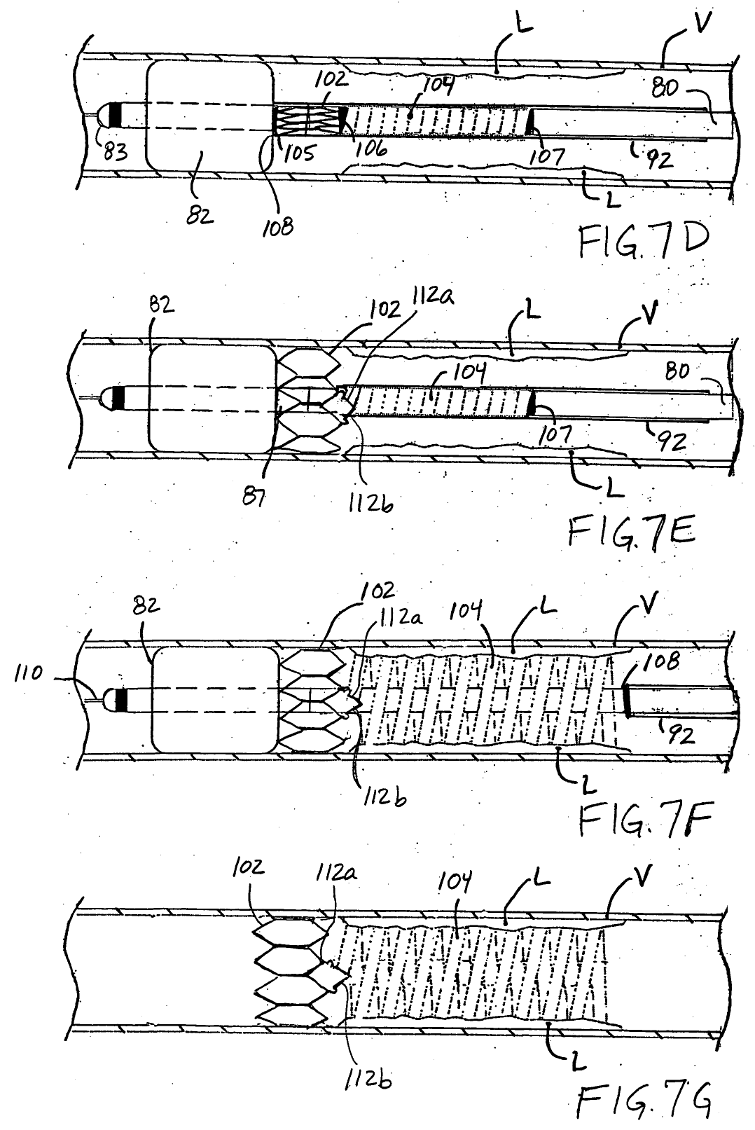

In addition, as described with respect to FIG. 7 hereinbelow, either before or after distal section 102 is expanded into engagement with the vessel wall, balloon 82 is expanded to contact the vessel wall. Balloon 82 therefore anchors distal end 83 of delivery catheter 90 relative to the vessel wall, so that no inadvertent axial displacement of the delivery catheter arises during proximal retraction of the sheath to release distal section 102 or helical section 104 of the vascular prosthesis 100.

With reference now to FIG. 7, a method of using delivery catheter 90 of FIG. 6 to perform angioplasty and deliver vascular prosthesis 100 of the present invention are described. Vascular prosthesis 100 is disposed in its delivery configuration with distal section 102 compressed around inner member 80 and retained by sheath 92. Distal section 102 of prosthesis 100 is disposed in contact with polymer layer 87 to prevent relative axial movement therebetween, as described above.

As shown in FIG. 7A, delivery catheter 90 is percutaneously and transluminally advanced along guide wire 110 until tip 83 of the catheter is disposed within lesion L within body vessel V, for example, as determined by fluoroscopic imaging. Once balloon 82 is positioned adjacent lesion L, sheath 92 is retracted proximally until radio-opaque marker 108 on sheath 92 is aligned with marker 105 of inner member 80, thereby indicating that the sheath has been retracted clear of balloon 82, as shown in FIG. 7B.

With respect to FIG. 7C, once balloon 82 is positioned adjacent lesion L, the balloon may be inflated to dilate a portion of the vessel and disrupt the plaque comprising lesion L. Balloon 82 then may be deflated, moved to another location within the lesion, and re-inflated to disrupt another portion of lesion L. This process is repeated until the lesion has been sufficiently disrupted to restore patency to the vessel.

Referring to FIG. 7D, after performing angioplasty, delivery catheter 90 is advanced so that balloon 82 is disposed adjacent healthy tissue, distal of the lesion. Balloon 82 then is inflated to engage the vessel wall and prevent axial displacement of the delivery catheter during subsequent retraction of sheath 92. Polymer layer 87 engages distal section 102 of vascular prosthesis 100, thereby preventing axial displacement of distal section 102 during retraction of sheath 92.

Referring to FIG. 7E, after balloon 82 is inflated to engage the vessel wall, sheath 92 is retracted proximally until distal section 102 self-expands into engagement with vessel wall within or distal to lesion L. Proximal movement of sheath 92 may be halted once radio-opaque marker 108 of sheath 92 is substantially aligned with radiopaque marker 106 of inner member 80. When released from the constraint provided by sheath 92, the struts of distal section 102 expand in a radial direction to engage the interior of vessel V. Stress relieving articulation, comprising connection members 112a and 112b, permit distal section 102 to engage into engagement with the wall of vessel V while mitigating torsional forces applied to the distal edge of helical section 104, in accordance with principles of the present invention.

Referring now to FIG. 7F, after distal section 102 is secured to the vessel wall distal of lesion L, sheath 92 is further retracted proximally to cause the helical section of stent 100 to unwind and deploy to its predetermined shape within vessel V. During proximal retraction of sheath 92, each subsequent turn unwinds one at a time and engages and conforms to an inner wall of vessel V in a controlled manner. Advantageously, torsional forces that are applied to distal section 102 during deployment of helical section 104 distributed through connection members 112a and 112b, and the associated hinges, over multiple cells of distal section 102, thereby reducing the risk of formation of inelastic strain or stress-induced fracture of the connection between distal section 102 and helical section 104.

In addition, any torsional forces applied to distal section 102 during retraction of sheath 92 are uniformly distributed over the surface of balloon 82, thereby reducing the risk of insult to the vessel endothelium. Once the last turn of the helical section of stent 100 is deployed, balloon 82 is deflated, and the sheath optionally may be advanced to cover balloon 82. Delivery catheter 90 then is withdrawn from the patient's vessel, and guide wire 110 is removed, completing the procedure.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

What is claimed is:

1. A vascular prosthesis comprising:
a radially self-expanding distal anchor;
a helical section comprising a plurality of turns;
a stress-relieving articulation coupling the radially self-expanding anchor to the helical section.

2. The vascular prosthesis of claim 1, wherein the radially self-expanding section comprises a zig-zag configuration defining a plurality of apices.

3. The vascular prosthesis of claim 2 wherein each apex of the zig-zag configuration comprises a pair of adjacent sections coupled by a bend.

4. The vascular prosthesis of claim 3, wherein the bend comprises a substantially “C”-shaped semicircular configuration.

5. The vascular prosthesis of claim 2 wherein the stress-relieving articulation comprises a first connection member, the first connection member comprising a substantially straight portion coupled to a first apex of the zig-zag configuration by a first hinge.

6. The vascular prosthesis of claim 5 wherein the substantially straight portion of the first connection member defines a distal edge of a final turn of the helical section.

7. The vascular prosthesis of claim 5, wherein the first hinge comprises a substantially “C”-shaped semicircular configuration.

8. The vascular prosthesis of claim 5 wherein the stress-relieving articulation further comprises a second connection member, the second connection member comprising a substantially straight portion coupled to a second apex of the zig-zag configuration by a second hinge, the second apex disposed adjacent to the first apex.

9. The vascular prosthesis of claim 8, wherein the second hinge comprises a substantially “C”-shaped semicircular configuration.

10. The vascular prosthesis of claim 8 wherein a proximal end of the first connection member is coupled to a proximal end of the second connection member by a third hinge.

11. The vascular prosthesis of claim 10, wherein the third hinge comprises a substantially “C”-shaped semicircular configuration.

12. A vascular prosthesis comprising:
a radially self-expanding distal anchor having a plurality of cells defined by a pair of zig-zag configurations joined by axially-oriented struts;
a helical section comprising a plurality of turns;
a stress-relieving articulation interposed between at least one of the plurality of cells and the the helical section.

13. The vascular prosthesis of claim 12, wherein each one of the plurality of cells includes a proximal apex.

14. The vascular prosthesis of claim 13 wherein proximal apex comprises a bend having a substantially “C”-shaped semicircular configuration.

15. The vascular prosthesis of claim 13 wherein the stress-relieving articulation comprises a first connection member coupled to a first proximal apex by a first hinge.

16. The vascular prosthesis of claim 15 wherein the first connection member comprises a substantially straight strut.

17. The vascular prosthesis of claim 15 wherein the first connection member comprises a distal edge of a final turn of the helical section.

18. The vascular prosthesis of claim 15, wherein the first hinge comprises a substantially “C”-shaped semicircular configuration.

19. The vascular prosthesis of claim 15 wherein the stress-relieving articulation further comprises a second connection member coupled to a second proximal apex by a second hinge, the second proximal apex disposed adjacent to the first proximal apex.

20. The vascular prosthesis of claim 19, wherein the second hinge comprises a substantially “C”-shaped semicircular configuration.

21. The vascular prosthesis of claim 19 wherein a proximal end of the first connection member is coupled to a proximal end of the second connection member by a third hinge.

22. The vascular prosthesis of claim 21, wherein the third hinge comprises a substantially “C”-shaped semicircular configuration.
